(19) United States
(12) Patent Application Publication
(10) Pub. No.: US 2008/0027305 A1
(43) Pub. Date: Jan. 31, 2008

(54) Automatic determination of tumor load

(76) Inventor: Lutz Gundel, Erlangen (DE)

Correspondence Address: HARNESS, DICKEY & PIERCE, P.L.C., P.O.BOX 8910, RESTON, VA, 20195, US

(21) Appl. No.: 11/882,154

(22) Filed: Jul. 31, 2007

(30) Foreign Application Priority Data
Jul. 31, 2006 (DE) 10 2006 035 617.9

(51) Int. Cl. A61B 5/00

(52) U.S. Cl. 600/407

(57) ABSTRACT

Embodiments generally relate to the automatic diagnosis of tumors. At least one embodiment of the invention relates to a medical appliance, to a method and/or to a computer program for automatically determining the tumor load from data records from imaging methods. According to at least one embodiment of the present invention, the medical appliance for determining the tumor load from data records from imaging methods includes at least one device/module for determining the size of lesions on the basis of given data records of imaging methods and tumor size determination criteria, at least one device/module for determining target lesions, at least one device/module for determining tumor loads on the basis of the determined lesion sizes of the target lesions and on the basis of tumor load criteria.

### PRIORITY STATEMENT

The present application hereby claims priority under 35 U.S.C. §119 on German patent application number DE 10 2006 035 617.9 filed Jul. 31, 2006, the entire contents of which is hereby incorporated herein by reference.

### FIELD

Embodiments of the invention generally relate to the automated diagnosis of tumors. Embodiments of the invention may relate in particular to a medical appliance, to a method and/or to a computer program for automatically determining the tumor load.

### BACKGROUND

In tumor diagnosis, data records from imaging methods, such as CT, MRT, ultrasound or PET/SPECT, and from combinations of these methods, are searched for tumors manually or with the assistance of CAD algorithms. If a tumor is found, then its size must be determined. For historical reasons, a measurement has been carried out using axial slices. Various criteria for determining the tumor size are used in practice. According to the so-called RECIST criterion, the maximum size measured in a slice is defined as the size. On the basis of the WHO criteria, this maximum extent is defined on the basis of the RECIST criterion together with the maximum extent at right angles to this, as the size. The measurement must be carried out for each tumor and represents a time-consuming task for the examiner. If there are a plurality of tumors, then, for example, a maximum of five tumors are defined per organ, and a representative sample for all of the organs affected is defined as so-called target lesions, which are subjected to further evaluation.

According to the RECIST criterion, the sum of the maximum extents of the target lesions, according to the WHO criteria, the sum of the products of the two maximum extents of the target lesions, represents the so-called tumor load, which is of very great importance for a therapeutic decision. For therapy purposes, a plurality of examinations are carried out at different times. The aim of this process is to find out whether the size of known tumors has changed as a result of the therapy. In this case, the tumor load is compared, with different comparison criteria being used in practice.

### SUMMARY

In at least one embodiment of the invention, a medical appliance, a method and/or a computer program is specified by which tumor diagnosis can be carried out on the basis of imaging methods, in a simple and cost-effective manner.

According to at least one embodiment of the present invention, the medical appliance for determining the tumor load from data records from imaging methods includes: at least one device/module for determining the size of lesions on the basis of given data records of imaging methods and tumor size determination criteria, at least one device/module for determining target lesions, and at least one device/module for determining tumor loads on the basis of the determined lesion sizes of the target lesions and on the basis of tumor load criteria.

The imaging methods that may be used include, for example, CT, MRT, ultrasound or PET/SPECT, with combinations of these methods or of the data records from these methods in general being possible. Different imaging methods produce data which in some cases relates to widely differing medical aspects. The expressions “tumor size” and “lesion size”, which are used synonymously and should be interpreted as the “tumor load” are used in a corresponding widespread form.

An aim of at least one embodiment of the present invention is to make changes in tumor tissues, such as necrotization, calcification or enrichment with local chemotherapeutics detectable and quantifiable. When using PET or SPECT methods, metabolism processes, and in the case of perfusion measurements, the vasculization of tumors, are detected and quantified. When using each of these methods, at least one embodiment of the present invention makes it possible to draw important conclusions about the disease in a simple manner and with little labor effort, that is to say in a cost-saving manner.

The device/modules for determining target lesions may be advantageously designed to determine the target lesions automatically or semi-automatically on the basis of criteria for determining target lesions.

The medical appliance advantageously has at least one lesion segmentation device/module for segmentation of lesions. The medical appliance is likewise advantageously equipped with at least one organ segmentation device/module for segmentation of organs which are affected by lesions. The at least one organ segmentation device/module is advantageously designed to additionally identify the organs and to produce the identification information obtained in the process. This makes it possible to delineate multiple tumors not only on the basis of their size but also on the basis of their position in an organ.

If the medical appliance is equipped with at least one lesion segmentation device/module and/or at least one organ segmentation device/module, it is advantageous for the at least one device/module for determining target lesions to be designed to determine the target lesions on the basis of the output from the at least one lesion segmentation device/module and/or on the basis of the output from the at least one organ segmentation device/module. It is also advantageous for the at least one device/module for determining target lesions to be designed to determine the target lesions on the basis of the lesion sizes determined by the at least one device/module for determining the size of lesions.

The various criteria are advantageously each defined interactively by a user, or are selected by a user from a selection of criteria stored in at least one memory device/module, or are read from a report, with the report preferably containing further anamnestic data, in particular previously created data records from imaging methods, or cross-references to such anamnestic data.

This makes it possible to use a plurality of criteria, such as the RECIST criterion, the WHO criteria, or user-defined criteria, such as hospital-specific, regionally different or research-specific criteria, flexibly, and nevertheless to handle these criteria easily and safely.

The medical appliance is advantageously designed to in each case store cross-references to the tumor size determination criteria used, to the tumor load criteria used, to the lesion sizes determined, to the definition of the target lesions, to the determined tumor loads, to the position information of lesions, and/or to the given data records or to the respective information itself, in a report.

The medical appliance advantageously has a first data interface for receiving position information which describes the positions of lesions in the given data records.

The medical appliance advantageously has at least one device/module for detecting lesions in the data records, with the at least one device/module for detecting lesions in the data records including at least one device/module for automatically detecting lesions and/or at least one device/module for manually detecting lesions.

The medical appliance advantageously has at least one recording device/module for recording first data records with second data records and at least one comparison device/module for automatically comparing lesion sizes and/or tumor loads from first data records with the corresponding lesion sizes and tumor loads from second data records, with the data records from imaging methods including the second data records.

These features may further simplify tumor diagnosis, in particular assessment of the progress of a disease or of a therapy, since these features may automate the detection of changes to lesion sizes and/or tumor loads.

The at least one comparison device/module may advantageously designed to read the criteria required for a comparison from a report, with the report preferably containing anamnestic data, in particular previously created data records from imaging methods, or cross-references to such anamnestic data.

The at least one recording device/module may be advantageously designed to determine first position information items which describe the positions of lesions that have been found in the first data records, in the second data records. In this case, it is advantageous for the lesions that have been found in the first data records, from which the first position information items have been determined, to be target lesions, and for the at least one recording device/module to be designed to read the definition of the target lesions from a report, with the report preferably containing further anamnestic data, in particular previously created data records from imaging methods, or cross-references to such anamnestic data.

The at least one device/module for determining the size of lesions may be advantageously designed to read tumor size determination criteria from a report and to determine the size of lesions in the second data records on the basis of the tumor size determination criteria that have been read, and the at least one device/module for determining tumor loads may be designed to read tumor load criteria from a report and to determine a tumor load on the basis of the lesion sizes, which have been determined for the second data records of the target lesions and on the basis of the tumor load criteria that have been read.

The at least one comparison device/module may be advantageously designed to read comparative data of the first data records from a report, with the report preferably containing further anamnestic data, in particular previously created data records from imaging methods, or cross-references to such anamnestic data. In this case, it is advantageous if the data records from imaging methods additionally comprise the first data records and if the at least one recording device/module may be designed to determine second position information items, which describe the positions of lesions which have been found in the second data records, in the first data records.

In the same way as the medical appliance described above, at least one embodiment of the invention also generally relates to a method for computer-aided determination of the tumor load from data records from imaging methods or using a computer program which is suitable for carrying out this method.

According to at least one embodiment of the present invention, the method for computer-aided determination of the tumor load from data records from imaging methods, includes: automatic determination of the size of lesions on the basis of given data records from imaging methods and tumor size determination criteria, determination of target lesions, automatic determination of one or more tumor loads on the basis of the determined lesion sizes of the target lesions and on the basis of tumor load criteria.

### BRIEF DESCRIPTION OF THE DRAWINGS

Embodiments of the invention will be explained in the following text using one example embodiment of the medical appliance and with reference to the attached figures, in which:

FIG. 1 shows a block diagram of some of the components of the example embodiment, and

FIG. 2 shows a block diagram of further components of the example embodiment.

### DETAILED DESCRIPTION OF THE EXAMPLE EMBODIMENTS

The terminology used herein is for the purpose of describing particular embodiments only and is not intended to be limiting of the present invention. As used herein, the singular forms “a”, “an” and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

Referencing the drawings, wherein like reference numerals designate identical or corresponding parts throughout the several views, example embodiments of the present patent application are hereafter described.

At least one example embodiment can carry out at least one of the following steps:
1. Searching for and measurement of the size of tumors in one or more data records from a first examination.
2. Measurement of the lesions found in the data records from the first examination at a later time during a subsequent examination, and determination of tumor growth or tumor reduction.
3. Searching for and measurement of the size of tumors in the subsequent examination, which tumors were found in the previous examinations.
4. Navigation to the locations of the newly found lesions in the data records from the first examination and checking whether they have been overlooked.

These steps and the example embodiment will be explained in more detail in the following text:

Step 1:

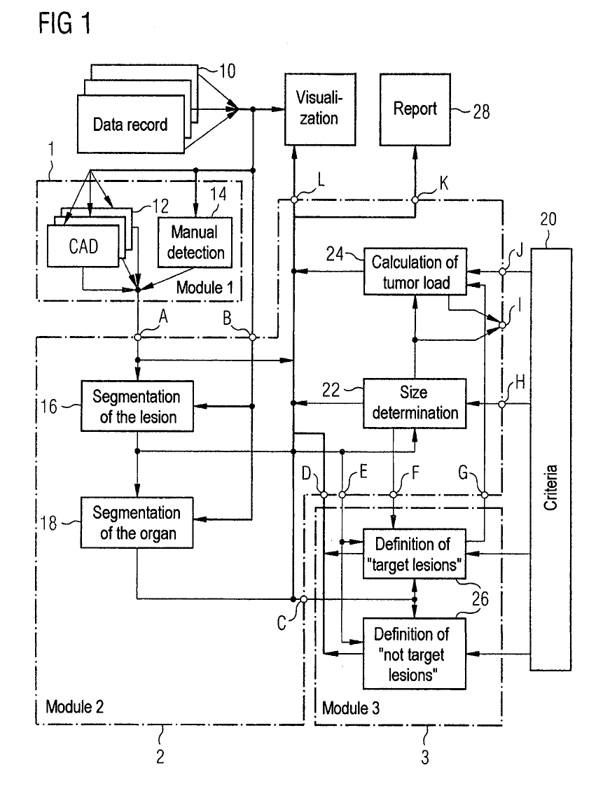

FIG. 1 shows those components of the example embodiment which are relevant for carrying out the first step. Tumor diagnosis can be carried out by the medical appliance for determining the tumor load by way of one or more data records 10. A plurality of data records 10 are obtained, for example, when an imaging method is carried out with and without contrast agent and relating to different phases (for example arterial, venous, late venous) of contrast agent flooding in the organs to be examined. It is advisable to use a number of methods in particular for an initial diagnosis, since different methods and different phases of the contrast-agent flooding respond differently to different tumors.

These data records 10 are displayed two-dimensionally or three-dimensionally to the examiner in order to manually search for supposed lesions or in order to check an automatic search. The search is carried out in a first module 1 of the appliance either manually by selection of specific data areas, with the examiner having the capability, for example, to scroll through a stack of multiplanar reconstructions, or automatically, for example by way of one or more Computer Aided Detection (CAD) algorithms. For this purpose, the example embodiment has at least one device/module 12 for automatic detection, which carry out the CAD algorithms, and has at least one device/module 14 for manual detection.

The position information associated with the lesions found is transmitted to a separate module 2 in the example embodiment via an interface A. There, based on the data records 10 which are transmitted to the second module via an interface B, the lesions found are segmented 16 by at least one lesion segmentation device/module 16, and the organs affected are segmented 18 by the at least one organ segmentation device/module 18. Imaging methods produce pixels arranged in three dimensions, so-called voxels. The at least one expression segmentation device/module is used for the association of a set of pixels with any given element of the body being imaged. The expression segmentation of a lesion therefore refers to the association of pixels relating to a given lesion, and the expression segmentation of an organ relates to the association of pixels with a given organ. In addition to pure segmentation, the at least one organ segmentation device/module 18 carries out an organ identification process. The at least one organ segmentation device/module 18 produces the identification information, so that the appliance knows which organ is affected by which lesion.

The lesion size is determined automatically, on the basis of criteria, by device/module 22 for size determination. The at least one device/module 22 for size determination are part of the second module 2 and obtain these size determination criteria via an interface H of the second module 2 from a criteria memory 20. The size determination criteria are either stored permanently in the criteria memory 20, for example in the form of a database, from where they can be selected by the examiner, or they can be interactively entered by the examiner, and stored there. Possible size determination criteria are, for example, the maximum diameter within a slice (RECIST criterion), and additionally the maximum diameter at right angles to this (WHO criteria), the maximum diameter within the volume, the volume itself or the ratio of the lesion volume to the organ volume. The interface I of the second module 2 makes the results of the size determination process available to further modules.

The appliance has at least one device/module 26 for determining target lesions, with the target lesions representing a subset of the tumors detected in the data records. The subset may be trivial, that is to say identical to the set of detected tumors. This depends on the patient data and the criteria used as the basis for determining the target lesions. The remaining lesions are associated with the class of non-target lesions. Only the target lesions are used to calculate the tumor load.

The target lesions may be defined manually, automatically or semi-automatically. If the target lesions are determined automatically or semi-automatically, the at least one 26 for determining target lesions obtain appropriate criteria from the criteria memory 20. These criteria for determining target lesions are either stored permanently in the criteria memory 20, for example in the form of a database, or can be entered interactively by the examiner, and stored there. Furthermore, if determined automatically or semi-automatically, the target lesions are determined on the basis of the segmentation results of the lesions and/or on the basis of the segmentation results of the organs affected, in particular the identification information and/or on the basis of the determined lesion sizes, for which purpose the example embodiment respectively provides the interfaces E, C and F of the second module 2. The criteria for determining target lesions are, for example, the five or ten largest lesions in the body, the five largest lesions of an organ, or lesions of a specific type. In the example embodiment, the means 26 for determining target lesions are in the form of a further, third module 3.

The criteria are used to determine the tumor load from the target lesions. The at least one device/module 24, provided for this purpose in the second module 2, for determining tumor loads obtain the tumor load criteria to be used via an interface J of the second module 2 from the criteria memory 20, and the definition of the target lesions, that is to say information as to which lesions represent target lesions, via an interface G of the second module 2.

Possible tumor load criteria are, for example, the sum of the maximum diameters based on the RECIST criterion (RECIST criterion), the sum of the products of the two maximum diameters based on the WHO criteria (WHO criteria), the sum of the lesion volumes or the sum of the percentages of the components of the lesion volumes with respect to the respective organ volumes. The tumor load criteria are either stored permanently in the criteria memory 20, for example in the form of a database, or can be selected from there by the examiner, or can be entered interactively by the examiner, and stored there. The interface I of the second module 2 makes the results of the tumor load determination process available to further modules.

The results of the individual functional units/devices/modules 12, 14, 16, 18, 22, 24, 26, in particular the position information relating to the detected lesions, the results of the lesion segmentation, the results of the organ segmentation, the lesion sizes determined, the definition of the target lesions, and the tumor load determined as well as the tumor size determination criteria used, the tumor load criteria and, derived from them or defined interactively, criteria for tumor growth and tumor reduction, in particular criteria for assessment of the change in the tumor load, as well as the path names of the data records 10 are stored in a report 28. The second module 2 provides the interface K for this purpose. The data stored in the report 28 is then available in machine-legible form for further examinations. Furthermore, if requested by the examiner, a doctor's brief can be generated from this, in which the diagnosis is written up in a form that can be read by people.

The appliance makes it possible to group criteria. In particular, grouping of in each case one representative of the tumor size determination criteria, the tumor load criteria, the criteria for tumor growth and tumor reduction, in particular the criteria for assessing the change in the tumor load, and criteria for determining target lesions, is possible and reduces the operator workload, since a single selection of a group such as this defines all the required criteria. Database techniques can be used to carry out the grouping and selection processes.

For display purposes, the patient data, that is to say the results of the functional units/devices/modules 12, 14, 16, 18, 22, 24, 26, are preprocessed, for example, in the form of a semi-transparent display. The display shows, in particular, the lesions found. In this case, it is possible to color the image on the basis of the categories target lesion/non-target lesion, size, tumor type or organ association, via the interface L of the second module 2.

Step 2:

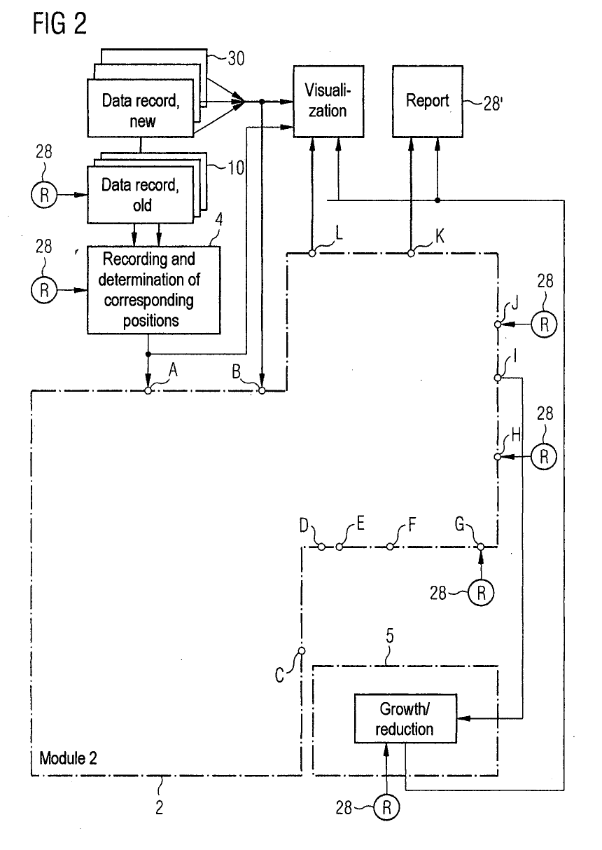

Steps 2, 3 and 4 will be explained with reference to FIG. 2. Like FIG. 1, FIG. 2 shows the second module 2. However, in order to allow concentration on further features, FIG. 2 did not show the individual functional units of the second module 2. The same reference symbols denote the same technical features.

As in the first examination, the new data record or records 30 is or are displayed for a subsequent examination, and is or are made accessible to the second module 2 via the interface B. The path names of the previously used data records 10 are read from the report 28, and are then loaded. A recording module 4 calculates an association between the voxels in the two data records, and reads the position information for the lesions found in the old data records 10, from the report 28. The positions of the lesions found in the old data records are therefore automatically determined in the new data records, and the process navigates to the positions of the lesions, in order to display them.

The information is made available to the second module 2 via the interface A. Segmentation of the lesions, segmentation of the organs, determination of the lesion size and calculation of the tumor load are carried out in the second module 2 by the at least one lesion segmentation device/module 16, the at least one organ segmentation device/module 18, the at least one device/module 22 for size determination of lesions and, respectively, the at least one device/module 24 for determining the tumor load. This is done in the same way as in the first examination with the difference that the tumor size determination criteria used, the tumor load criteria and the definition of the target lesions are read from the report 28 via the respective interfaces H, J and G.

The lesion sizes are read from the second module 2 via the interface I in a comparison module 5, where they are automatically compared with the lesion sizes of the data records from the first examination. For this purpose, the lesion sizes from the first examination are read from the report 28. Any criteria required for comparison purposes are likewise read from the report 28. The changes in the size of individual tumors are displayed, for example in a colored form in an overview. The tumor load is likewise read from the second module 2 via the interface I and is automatically compared there with the tumor load, read from the report 28, from the data records from the first examination.

The criteria required to assess in the tumor load are read from the report 28. Possible criteria for this purpose are, for example, the RECIST criterion and the WHO criteria. Based on the RECIST criterion, a tumor load reduction of more than 30 percent is assessed as a significant improvement, a tumor load increase of more than 20 percent is assessed as a significant deterioration, and values between these are assessed as a stable state. Based on the WHO criteria, a tumor load reduction of more than 50 percent is assessed as partial response to the therapy, a reduction of 25 percent is assessed as a minor response to the therapy, and increase of more than 50 percent is assessed as a progressive disease, and values in between are assessed as a stable state.

Furthermore, the module detects tumors which have disappeared as a result of therapy, and tumors which have newly occurred. The results of the comparison module 5 are stored in a report 281. The report 28 in which the results of the subsequent examination are stored may be the report 28 in which the results of the first examination were stored, or may be different from it. In the case of two reports, it is advantageous for the reports to continue cross-references to one another, or for the earlier report 28 to contain a cross-reference to the later report 28′.

Step 3:

After step 2, the new data records 30 are searched for new lesions which, if appropriate, are measured. The results are stored in the report 28′. This is all done as in step 1, with the only differences being that, when lesions are searched for manually or automatically using the first module 1, information about lesions that are already known is inserted, so that only new findings are reported to the second module 2 via the interface A, and that the tumor size determination criteria to be used need not be determined again, since they are already defined.

Step 4:

If new lesions have been found in step 3, then a check is now carried out to determine whether they were overlooked in the original examination. This is done in an equivalent manner to step 2, but with the role of the old data records 10 and of the new data records 30 being interchanged. The recording need not be carried out again, since it is possible to make use of the recording results from step 2. The positions at whose locations new lesions have been found in the new data records 30 are indicated in the old data records 10. If there are no lesions there, as can once again be decided manually or automatically, thus, no tumors were overlooked. Otherwise, the growth of the lesions is determined, and is stored in the report 28′.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDS; magneto-optical storage media, such as MOs; magnetism storage media, including but not limited to Floppy Disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A medical appliance for determining tumor load from at least one data record of at least one imaging method, comprising:
means for determining a size of lesions on the basis of the at least one data record and tumor size determination criteria;
means for determining target lesions; and
means for determining tumor loads on the basis of the determined lesion sizes of the target lesions and on the basis of tumor load criteria.

2. The medical appliance as claimed in claim 1, wherein the means for determining target lesions is designed to determine the target lesions at least one of automatically and semi-automatically on the basis of criteria for determining target lesions.

3. The medical appliance as claimed in claim 1, further comprising:
lesion segmentation means for segmentation of lesions and organ segmentation means for segmentation of organs affected by lesions.

4. The medical appliance as claimed in claim 3, wherein the organ segmentation means are additionally designed to identify the organs and to produce the identification information obtained in the process.

5. The medical appliance as claimed in claim 3, wherein the means for determining target lesions is designed to determine the target lesions on the basis of the output from the lesion segmentation means and to show at least one of on the basis on the output from the organ segmentation means and on the basis of the lesion sizes determined by the means for determining the size of lesions.

6. The medical appliance as claimed in claim 1, wherein the various criteria are each at least one of defined interactively by a user and selected by a user from a selection of criteria a least one of stored in a memory and read from a report.

7. The medical appliance as claimed in claim 1, wherein cross-references at least one of to the tumor size determination criteria used, to the tumor load criteria used, to the lesion sizes determined, to the definition of the target lesions, to the determined tumor loads, to position information of lesions and to at least one of the at least one data record and the respective information are stored in a report.

8. The medical appliance as claimed in claim 1, further comprising:
a first data interface for receiving position information which describes the position of lesions in the at least one data record.

9. The medical appliance as claimed in claim 1, further comprising,
means for detecting lesions in the at least one data record, including at least one of means for automatically detecting lesions, and means for manually detecting lesions.

10. The medical appliance as claimed in claim 1, further comprising:
recording means for recording first data records with second data records and comparison means for automatically comparing at least one of lesion sizes and tumor loads from first data records with at least one of the corresponding lesion sizes and tumor loads from second data records, with the data records from at least one imaging method comprising the second data records.

11. The medical appliance as claimed in claim 10, wherein the comparison means are designed to read the criteria required for a comparison from a report.

12. The medical appliance as claimed in claim 10, wherein the recording means are designed to determine first position information items, which describe the positions of lesions which have been found in the first data records in the second data records.

13. The medical appliance as claimed in claim 12, wherein the lesions which have been found in the first data records and from which the first position information items have been determined are target lesions, the recording means being designed to read the definition of the target lesions from a report.

14. The medical appliance as claimed in claim 10, wherein the means for determining the size of lesions are designed to read tumor size determination criteria from a report and to determine the size of lesions in the second data records on the basis of the tumor size determination criteria that have been read, and wherein the means for determining tumor loads are designed to read tumor load criteria from a report and to determine a tumor load on the basis of the lesion sizes, determined for the second data records of the target lesions and on the basis of the tumor load criteria that have been read.

15. The medical appliance as claimed in claim 10, wherein the comparison means are designed to read comparative data of the first data records from a report.

16. The medical appliance as claimed in claim 15, wherein the data records from imaging methods additionally comprise the first data records and wherein the recording means are designed to determine second position information items, which describe the positions of lesions which have been found in the second data records, in the first data records.

17. A method for computer-aided determination of one or more tumor loads from at least one data record from at least one imaging method, the method comprising:
automatically determining a size of lesions on the basis of the at least one data record from at least one imaging method and tumor size determination criteria;
determining target lesions; and
automatically determining the one or more tumor loads on the basis of the determined lesion sizes of the target lesions and on the basis of tumor load criteria.

18. The method as claimed in claim 17, wherein the determining of the target lesions is carried out at least one of automatically and semi-automatically, at least partially on the basis of criteria for determining target lesions.

19. The method as claimed in claim 17, further comprising the automatic segmentation of lesions and automatic segmentation of organs which are affected by lesions.

20. The method as claimed in claim 19, wherein, in the automatic segmentation of organs, the organs are additionally identified, and the identification information obtained in this way is produced.

21. The method as claimed in claim 19, wherein, in the determining of the target lesions, the target lesions are at least partially determined at least one of on the basis of the results of the automatic segmentation of lesions, at least partially on the basis of the results of the automatic segmentation of organs, and at least partially on the basis of the results of the automatic determination of the size of lesions.

22. The method as claimed in claim 17, wherein the various criteria are at least one of each defined interactively by a user and selected by a user from a selection of criteria stored in a memory, and read from a report.

23. The method as claimed in claim 17, wherein cross-references to at least one of the tumor size determination criteria used, the tumor load criteria used, the lesion sizes determined, the definition of the target lesions, the tumor loads determined, position information for lesions and the at least one data record and the information itself are stored in a report.

24. The method as claimed in claim 17, further comprising:
determining position information which describes the positions of lesions in the at least one data record.

25. The method as claimed in claim 24, wherein the determining position information is carried out at least partially by detection of lesions in the at least one data record, in which case the detection process is carried out at least one of automatically and manually.

26. The method as claimed in claim 17, further comprising:
recording first data records with second data records; and
automatically comparing at least one of lesion sizes and tumor loads from the first data records with the corresponding at least one of lesion sizes and tumor loads from the second data records, with the data records from at least one imaging method comprising the second data records.

27. The method as claimed in claim 26, wherein the criteria required for a comparison are read from a report.

28. The method as claimed in claim 26, further comprising:
automatically determining first position information, which describes the positions of lesions that have been found in the first data records, in the second data records.

29. The method as claimed in claim 28, wherein the lesions that have been found in the first data records, from which the first position information is determined, are target lesions, with the definition of the target lesions being read from a report.

30. The method as claimed in claim 26, wherein, in the automatically determining the size of lesions, tumor size determination criteria are read from a report and the size of lesions in the second data records are determined on the basis of the tumor size determination criteria that have been read, and wherein, in the step of automatically determining one or more tumor loads, tumor load criteria are read from a report and a tumor load is determined on the basis of the lesion sizes, that have been determined for the second data records, of the target lesions and on the basis of the tumor load criteria that have been read.

31. The method as claimed in claim 26, further comprising:
reading data to be compared in the first data records from a report, the report containing previously created data records from imaging methods, or cross-references to anamnestic data.

32. The method as claimed in claim 31, further comprising:
automatically determining the second position information items, which describe the positions of lesions that have been found in the second data records, in the first data records, with the data records from imaging methods additionally comprising the first data records.

33. A computer program which, when loaded in the main memory of a computer, is suitable for carrying out the method for computer-aided determination of the tumor load from data records from the method as claimed in claim 17.

34. The medical appliance as claimed in claim 2, further comprising:
lesion segmentation means for segmentation of lesions and organ segmentation means for segmentation of organs affected by lesions.

35. The medical appliance as claimed in claim 34, wherein the organ segmentation means are additionally designed to identify the organs and to produce the identification information obtained in the process.

36. The medical appliance as claimed in claim 11, wherein the report contains anamnestic data.

37. The medical appliance as claimed in claim 36, wherein the report contains at least one of particular previously created data records from at least one imaging method, and cross-references to the anamnestic data.

38. The method as claimed in claim 18, further comprising the automatic segmentation of lesions and automatic segmentation of organs which are affected by lesions.

39. The method as claimed in claim 22, wherein the report contains anamnestic data.

40. The method as claimed in claim 39, wherein the report contains at least one of particular previously created data records from at least one imaging method, and cross-references to the anamnestic data.
